(19) United States
(12) Patent Application Publication
(10) Pub. No.: US 2024/0407645 A1
(43) Pub. Date: Dec. 12, 2024

(54) METHOD OF POST-OPERATIVE VISION SIMULATION AND SYSTEM IMPLEMENTING THE SAME

(71) Applicant: KOC UNIVERSITESI, Istanbul (TR)

(72) Inventors: Hakan UREY, Istanbul (TR); Koray KAVAKLI, Istanbul (TR); Gunes AYDINDOGAN, Istanbul (TR); Afsun SAHIN, Istanbul (TR)

(73) Assignee: KOC UNIVERSITESI, Istanbul (TR)

(21) Appl. No.: 18/699,349

(22) PCT Filed: Oct. 8, 2021
(86) PCT No.: PCT/TR2021/051029

§ 371 Date: Apr. 8, 2024

(51) Int. Cl. A61B 3/113; A61B 3/00

(52) U.S. Cl. CPC A61B 3/113; A61B 3/0025; A61B 3/005

(57) ABSTRACT

A near-eye ophthalmic simulation device suitable for use before cataract and/or refractive lens exchange operations is provided, including a pupil tracking unit to detect the position of an eye pupil, a computer-generated holographic display that can form at least one exit pupil wherein the each of the at least one exit pupils are independently sized and positioned relative to the position of said eye pupil; wherein each of the at least one exit pupil is configured to create a projected pattern on the retina of the viewer and carries visual information configured to simulate post-operative vision, wherein at least one visual parameter is selectively configurable within said simulator.

### CROSS REFERENCE TO THE RELATED APPLICATIONS

This application is the national phase entry of International Application No. PCT/TR2021/051029, filed on Oct. 8, 2021, the entire contents of which are incorporated herein by reference.

### TECHNICAL FIELD

The invention presented hereby generally concerns methods of determining the correct intraocular lens type to be used for surgical lens replacement. Disclosed invention more specifically relates to methods and systems used for assessing subjective post-operative vision and implementation of bespoke intraocular lenses (IOL) for cataract and refractive lens exchange (RLE) surgery candidates.

### BACKGROUND

Cataract is an ophthalmic disease in which a cloudy area is formed in the lens of the eye, resulting in blurred vision. Cataract is the world's most common cause of preventable blindness and is responsible for the loss of vision of 50 million people. Currently, primary prevention or medical treatment options are lacking, making surgical removal and replacement of eye lens the only treatment option. More than 100 million cataract surgeries (the most common surgery in the world) are performed each year mostly in developed countries. Furthermore, there are also people who would like to go through Refractive Lens Exchange (RLE) surgery, in order to improve their vision and eliminate the need for eyeglasses even though they don't suffer from cataracts, where patients generally being the ages of 40 and 60.

Cataract surgery evolved significantly within the last decade with several Intraocular Lens (IOL) options being available for cataract surgery. Since there are a wide variety of IOLs available in the market, it gets ever more difficult to match the right patient with the right type of IOL suitable to them. This technical problem itself relies on issues that need to be addressed, such as the level of possible visual acuity improvement in patients, whether they will stop using eyeglasses if they opt for IOLs, and whether they will experience side effects such as halo, glare around light sources and dysphotopsia. As such, there is a strong need for a vision simulator which will help patients visualize the post-operative performance of different IOL options before cataract surgery.

In the state of the art, there exist several imaging devices for diagnosis of cataract and pre-operative evaluation such as optical coherence tomography (OCT), adaptive optics imaging systems (aberrometry), cataract densitometer, corneal topography. However, those options do not work through dense hypermature cataractous lenses due to large scattering. Moreover, none of those imaging modalities can predict potential visual acuity and which IOL type is best for a given particular patient. For cataract patients, potential acuity meters (PAM) measure the visual acuity (VA) that the patient is likely to achieve once there are no more opacifications of the ocular media of the eye. The purpose of such devices is to detect patients who will not benefit from cataract surgery; for example, those with macular problems or neurological issues. To separate visual loss due to opacifications from other reasons, the method used for PAM devices takes advantage of the fact that the opacification is typically not homogeneous and there are typically some small, relatively clear areas in the otherwise opaque lens. These clear areas are used to project a target (numbers, letters or stripes) onto the retina using a narrow light beam. The method requires strong user cooperation to align the small beam with the desired section of the patient's pupil.

A pinhole occluder is an opaque disk with one or more small holes through it, used by ophthalmologists to test visual acuity. The occluder is a simple way to focus light, as in a pinhole camera. This can be used to distinguish visual defects caused by refractive error, which improve when the occluder is used, from other problems, which do not. Also, there is not enough light due to the small pinhole, pinhole size is not adjustable, not steerable (no eye tracker), field-of-view (FOV) is narrow since pinhole glass is 10-12 mm ahead of the cornea corresponding to a circular field of vision of about 4 degrees. Therefore, pinhole occluders are not very effective for cataract patients.

As such, said devices are limited to assessing problems with the retina such as age-related macular degeneration and require strong patient-doctor cooperation, with tests often being time-consuming. Currently there is no simulator to simulate and assess the performance of trifocal IOLs before surgery.

A recent study by Vinas et al. titled “Visual simulators replicate vision with multifocal lenses” compares performance of real IOLs, spatial light modulator-based adaptive optics simulators, and opto-tunable lens-based adaptive simulators using a phantom eye (i.e., artificial eye). Such adaptive optics simulators are useful as a research device and can be effective in replicating the performance of multifocal IOLs using an artificial eye. However, such simulators are not suitable for subjective evaluations with patients.

Cataract surgery can be performed only once, is irreversible, and carries high-risk as it impacts the most vital sense, the vision. Selection of suitable IOLs is difficult for both doctors and patients. There exists an objective need to develop a diagnostic device for use before cataract surgeries to help match the right patient to right IOL and reduce the anxiety of both patients and doctors.

### Objects of the Present Invention

Primary object of the disclosed invention is to present a method of in vivo post-operative vision simulation before lens replacement surgeries for cataract and other optic ailments.

Another object of the disclosed invention is to present a method of post-operative vision simulation with the aid of a computational holographic display and pupil tracker.

Another object of the disclosed invention is to present a method of post-operative vision simulation whereby IOL selection module is comprised to guide cataract patients about IOL options and corresponding visual performance, which will lead to better expectation management and shortening decision times by more than 50%.

### SUMMARY

Present invention discloses a novel method that addresses aforementioned problems using CGH display technology, which enables to shape and steer the beam of light through the relatively clear areas of the cataractous lens of a patient who is a candidate for lens replacement procedure. A programmable exit pupil and holographic pupil formation help patients see crisp images even through cataractous lenses. One can form single or multiple pupils, which help create images at different focal depths simultaneously without a conflict between the vergence and accommodation (VAC).

Eye box steering method in the disclosed invention allows scanning capabilities over the occluded parts of the pupil and detects as well as directs the optical beam through the non-cataractous regions on the crystalline lens. The adjustable depth of holographic displays allows to test accommodation response and the virtual image can be replaced at the desired depth to correct the refractive errors.

Disclosed invention incorporates a pupil tracker and automatic algorithmic adjustments using patient's diagnostics data, reducing the measurement times, and making the technology more accessible for patients. Once the device is aligned and the position is calibrated with the aid of the pupil tracker cameras and inter-pupillary distance adjustments, multiple small regions on the pupil were dynamically addressed, through which virtual images are sent to the retina, granting a unique ability to make use of non-cataractous parts of the lens efficiently.

The holographic beam can also incorporate correction for phase errors as well as scattering in the optical train, an iterative algorithm can be used to improve the retinal image by changing the hologram displayed on the spatial light modulator. Inverse of the scattering model of the medium and the inverse of the aberrations can be computed and incorporated in the CGH calculation.

### BRIEF DESCRIPTION OF THE DRAWINGS

Accompanying figures are given solely for the purpose of exemplifying a a method and system of in vivo post-operative vision simulation using CGH, whose advantages over prior art were outlined above and will be explained in brief hereinafter.

The figures are not meant to delimit the scope of protection as identified in the claims nor should they be referred to alone in an effort to interpret the scope identified in said claims without recourse to the technical disclosure in the description of the present invention.

FIG. 1 illustrates a head-worn CGH display module, which can show virtual images at different depths.

FIG. 2 illustrates at least one, preferably two exit pupils configured to correspond to the relatively clear areas of the cataractous lens.

FIG. 3 illustrates holographically formed virtual objects corresponding to near, intermediate and far vision for a monofocal lens, natural or IOL.

FIG. 4 illustrates holographically formed virtual objects being superimposed corresponding to near, intermediate and far vision for a trifocal lens IOL.

FIG. 5 illustrates a case where near, intermediate and far holographic virtual images are generated based on a visual acuity test patterns.

FIG. 6 illustrates virtual images formed at different distances from the eye using multiple exit pupils with a computer generated holographic (CGH) display architecture.

FIG. 7 illustrates holographic display and pupil tracker architecture.

FIG. 8 illustrates a benchtop device according to an embodiment of the disclosed invention.

FIG. 9 demonstrates a block diagram of the post-operative vision simulation method as set forth by the disclosed invention.

### DETAILED DESCRIPTION OF THE EMBODIMENTS

10) Eye
11) Retina
12) Cornea
13) Eye pupil
14) Exit pupil
   a. Exit pupil one
   b. Exit pupil two
   c. Exit pupil three
15) Exit pupil plane
16) Exit pupil beam
17) Diffraction order
18) Head-worn device
19) Computation unit
20) Virtual image
21) Computer generated holographic display
22) Pupil tracker camera
   a. Visible pupil tracker camera
   b. Near-infrared pupil tracker camera
23) Near object
24) Intermediate object
25) Far object
26) Focussed image
27) Defocussed image
28) Trifocal IOL
29) Real eye
30) Artificial eye
31) Scattered ray
32) Visual artefact
   a. Halo
   b. Glare
33) Monofocal lens
34) NIR LED
35) Phase modulated waveform
36) Beam splitter
37) Pupil tracker unit
38) Point light source,
39) Spatial light modulator (SLM)
40) Optical components
41) Benchtop simulator device
42) Head rest
43) Mechanical adjustments
44) Perspective hologram

The present invention discloses a post-operative vision simulator for candidates of cataract or refractive lens exchange (RLE) surgery. The proposed instrument as part of the present disclosure is a holographic display integrated with a real-time pupil tracker and transmits a holographic image through the patient's pupil. Said instrument incorporates programmable diffractive optical components to digitally control the size and location of the light beams that are entering through the patients' pupils.

Present invention also discloses a novel method that addresses aforementioned problems using CGH display technology. Disclosed invention enables to shape and steer the beam of light through the relatively clear areas of the cataractous lens. A programmable exit pupil and holographic pupil formation help patients see crisp images even through cataractous lenses. One can form single or multiple pupils, which help create images in different focal depths using the multiple exit pupil principle.

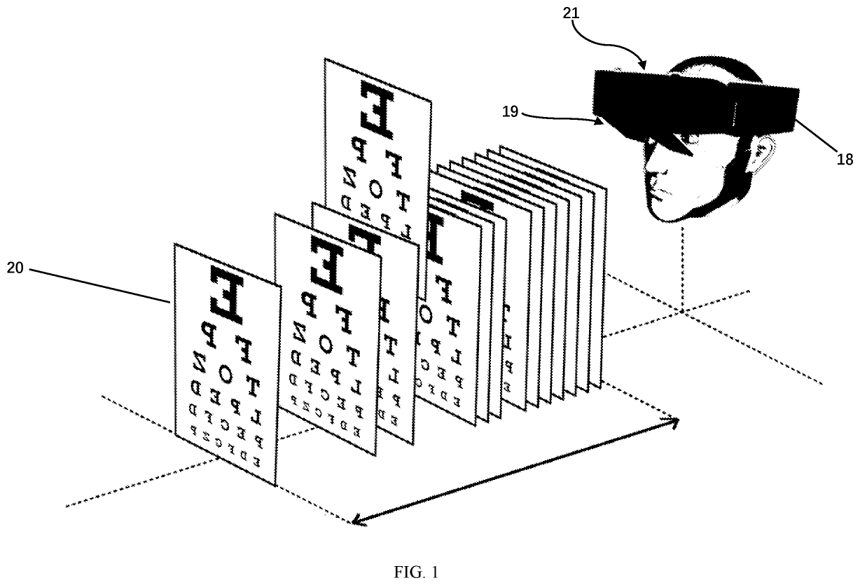

FIG. 1 demonstrates a head-worn device (18) comprising a CGH display (21) module according to at least one embodiment of the disclosed invention. According to several embodiments, said CGH display (21) module is configured to display relevant predetermined sets of images to the user wearing it. One such set of images may comprise Snellen chart images, while another set may comprise LogMAR chart images. According to different embodiments, said images may correspond to images representing different focal distances, i.e. near, intermediate, and far objects (23, 24, 25).

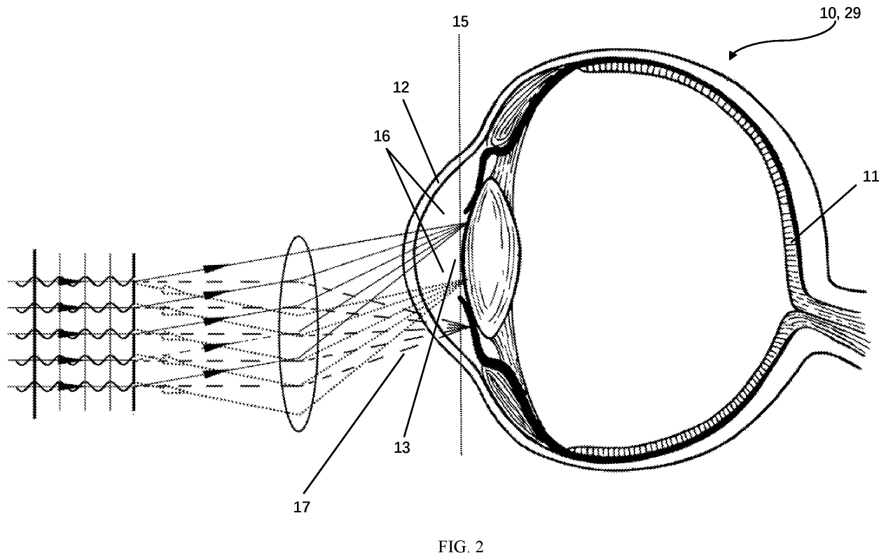

FIG. 2 demonstrates the configuration wherein at least one exit pupil beams (16) that are generated by the algorithm based on the oculography data of the patient determining the more viable and less dense sections of the cataractous lens. Disclosed invention utilizes said exit pupil beams (16) for creating images in different focal depths. In different embodiments of the disclosed invention, said exit pupils may correspond to at least 0^{th}, 1^{st} and 2^{nd} diffraction orders (17, 17a, 17b, 17c) for a case which enables different orders on a multifocal lens to be realized holographically. One such case may be a trifocal lens (28), suitable for replacing a cataractous lens via surgery. In other embodiments, multiple exit pupils (14) are formed, such as two and three. This enables the disclosed invention to use CGH through multiple different, non-identical however relatively less mature regions on the cataractous lens for forming exit pupils.

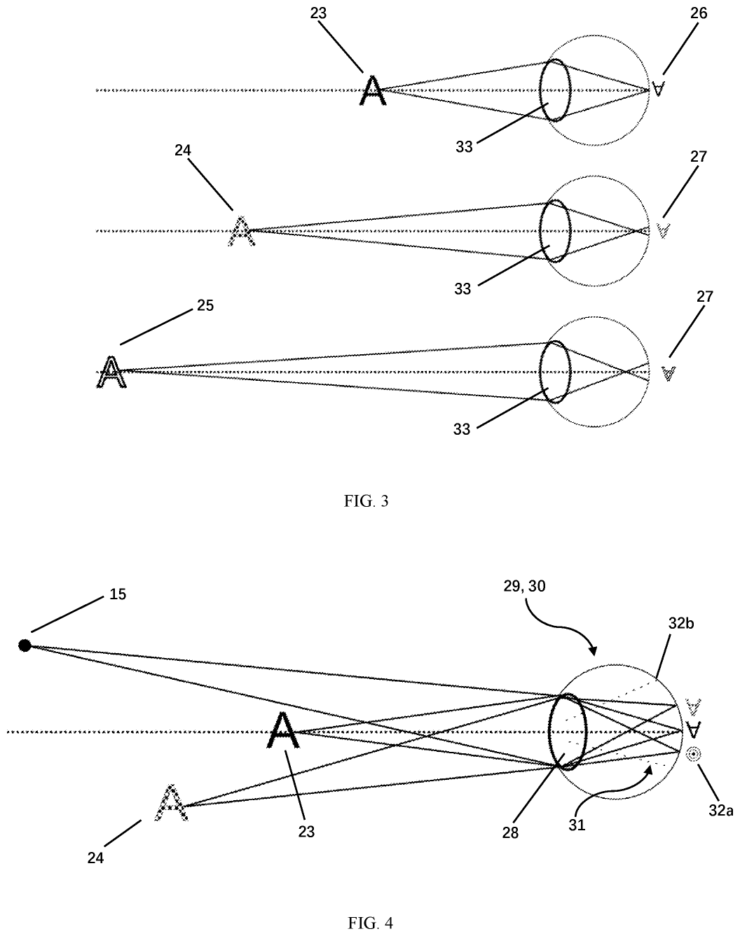

FIG. 3 demonstrates images generated for targeting different focal depths, namely near, intermediate and far vision using a monofocal lens designed to have near objects (23) as focused image (26) and the others as defocused images (27). Disclosed invention is as such capable of simulating conditions for at least a monofocal lens (33), which is analogous to the general properties of a normal, organic lens that is found by default in humans. Such focal depths, inasmuch as their difference is a result of spatial positioning in a real world case, may be holographically generated such that they refer to at least one near object (23), an intermediate object (24) and a far object (25).

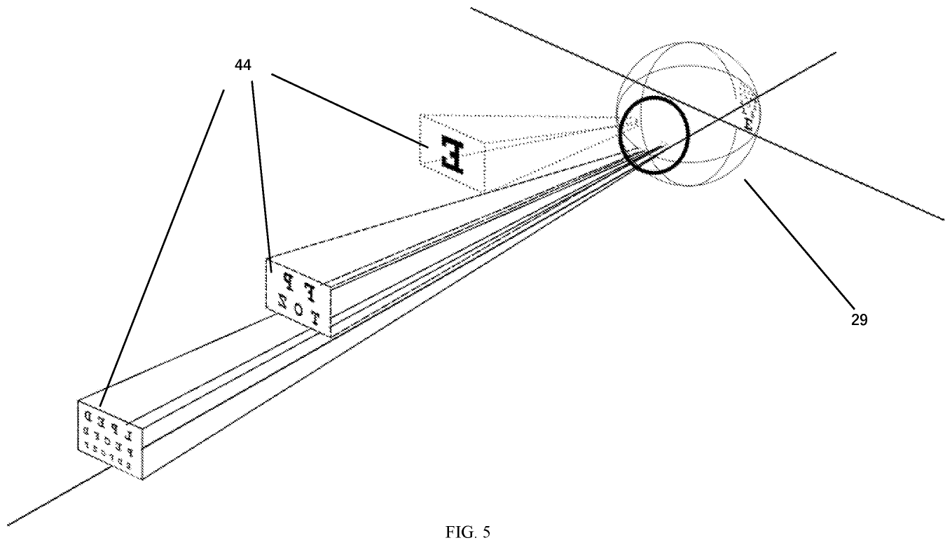

FIG. 4 demonstrates images generated for targeting different focal depths, namely near, intermediate and far vision superimposed and all are focused images (26). A bright light source located at far distance is illustrated in FIG. 4. The corresponding retinal image can have a ring like colored artefacts, i.e., halo (32.a) visual artefacts around the image of the bright light source. Furthermore, the diffractive multifocal or trififocal IOLs (28) or cataractous lenses can create varying degrees of scattered light and background glare (32.b).

FIG. 5 demonstrates generation of virtual images in the form of perspective holograms (44) at different depths, one embodiment of such using said image generation based on CGH with respect to visual acuity tests known in the practice. In several embodiments, such a visual acuity test may take advantage of a LogMAR chart or Snellen chart. Different depth images are configured to correspond to upper, middle and lower sections of a LogMAR chart or Snellen chart.

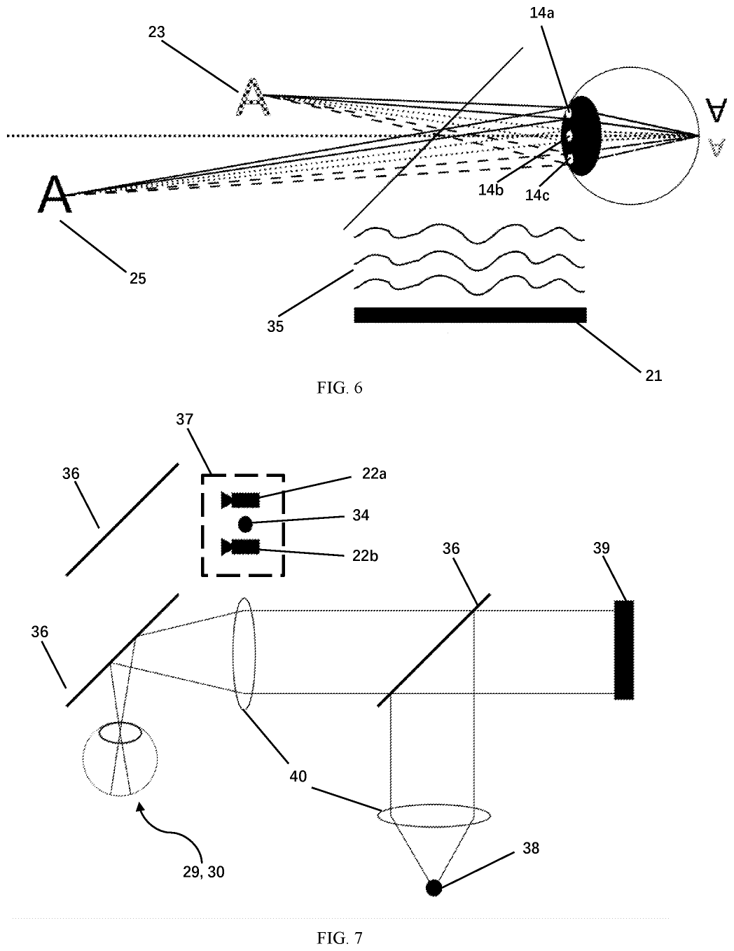

FIG. 6 illustrates virtual images formed at different distances from the eye using a CGH display (21) architecture. There are 3 exit pupils (14.a, 14.b, 14.c) formed across the eye pupil (13) in the illustration. Each exit pupil (14) can carry visual information corresponding to the same or different perspective views of a scene, i.e., perspective holograms (44). Each perspective hologram (44) can be 2D (planar) or 3D (volumetric). When different views are relayed to the retina through different exit pupil (14), then the combined image would be a multifocus image, i.e., virtual images (20) at different depths will appear as focused images (26). Different perspective images can create non-overlapping or overlapping images on the retina and appear at a predetermined focus distance determined by the CGH. This is similar to the operation of trifocal IOLs, which can have three distinct focal distances simultaneously. Even though multifocal IOLs are not able to accommodate, objects at different distances such as near, intermediate, and far can carry different beam energy corresponding to a certain defocus curve and appear in focus simultaneously. Other interim distances will appear blurred. In the CGH display, it is possible to selectively adjust which distances appear in focus and which ones appear out-of-focus or blurred using algorithms.

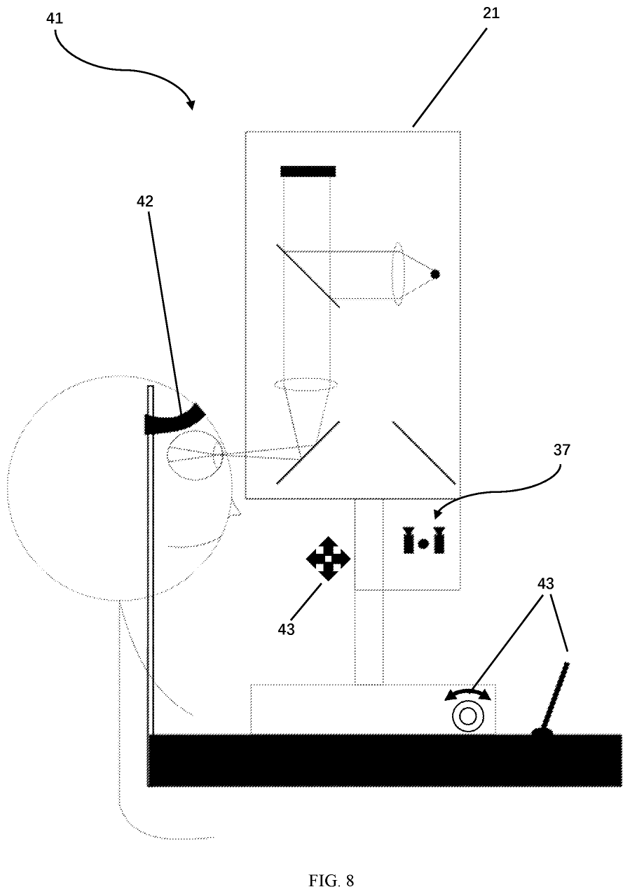

FIG. 7 illustrates the CGH display (21) unit and the pupil tracking unit (37) housing a visible camera (22a) and near infrared camera (NIR) (22b). NIR camera can detect eye pupil (13) position, determine its size, and align it with the other data such as Scheimpflug cataract densitometer data. Visible camera (22a) can detect the 0^{th} and other diffraction orders (17) using corneal reflections from the regions outside the eye pupil (13) or using purkinje reflections. Those can be used to align the CGH generated beams with the pupil of the viewer. The optical unit can also include an additional beam splitter to make the outside world visible such that virtual objects and real objects can be seen together simultaneously. CGH display architecture can employ a number of different optical architectures. It can be in Fourier hologram configuration, the SLM (39) can have collimated, converging, or diverging beam illumination. In another embodiment, CGH display (21) can use the optical relay lenses used in augmented reality or virtual reality glasses architecture such as waveguides, freeform mirrors and lenses.

FIG. 8 illustrates an ophthalmic benchtop simulator device (41) housing the CGH display (21) unit, pupil tracking unit (37), mechanical adjustments for aligning the left and right eye pupils (13) of the user or patient with the exit pupil plane (15) of the beanchtop simulator device (41). The user's head can be immobilized with respect to the device.

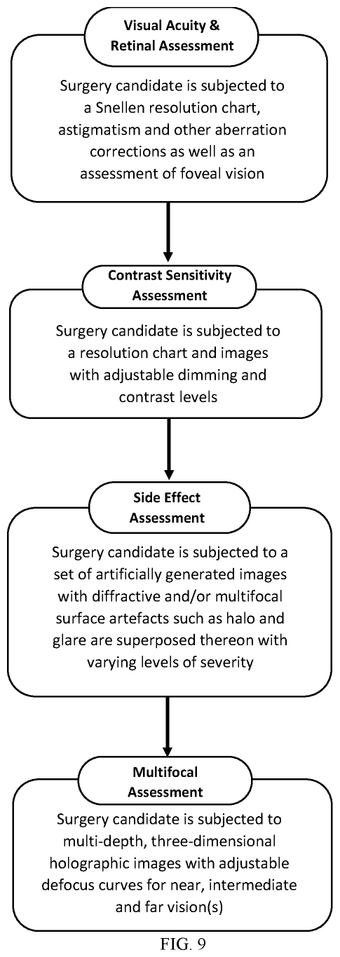

FIG. 9 demonstrates a block diagram of the post-operative vision simulation method as set forth by the disclosed invention. Said method comprises steps of; multifocal assessment, whereby a person is subjected to multi-depth, three-dimensional holographic images with adjustable defocus curves for near, intermediate and far vision(s), visual acuity and retinal assessment, whereby a person is subjected to a visual acuity chart, astigmatism and other aberration corrections as well as an assessment of foveal vision, contrast sensitivity assessment, whereby a person is subjected to a visual acuity chart and images with adjustable dimming and contrast levels, side-effect assessment, whereby a person is subjected to a set of artificially generated images with diffractive and/or multifocal surface artefacts such as halo and glare are superposed thereon with varying levels of severity, and multifocal assessment, whereby a person is subjected to multi-depth, three-dimensional holographic images with adjustable defocus curves for near, intermediate and far vision(s).

In another configuration, the device can be made binocular and present holographic stereograms to the viewer to stimulate 3D vision.

In another embodiment, different views sent through different exit pupils (14, 14a, 14b, 14c) can be different color (or wavelengths). Main benefit of multiplexing with different colors is the reduced frame rate requirement from the SLM (29). This scheme also avoids interference between the exit pupils (14, 14a, 14b, 14c) at the retina (11). Such interference could cause undesired fringing artefacts and noise-like appearance in the viewed images and can reduce the resolution. It is possible to add different phases to each exit pupil beam (16) to reduce and eliminate such undesired interference and speckle artefacts.

Main benefit of time-multiplexing different views is to reduce the possible coherent artifacts such as interference and speckle between the exit pupils (14, 14a, 14b, 14c). In another embodiment, exit pupil beams (16) can be formed in a time-sequential manner. Such a time-multiplexing scheme may also create a stationary image on the fovea while the other beams forming at different parts of the retina (11) can appear to be flickering if the refresh frequency is not higher than what could be detected by the eye (10). When the gaze of the person moves to content at other directions, then those become static while those that are at different depths can appear flickering.

Disclosed invention employs a pupil tracking unit (37), which will automatically find the relatively clear areas of the cataractous lens using the visible camera (22a), near infrared camera (22b) and/or cataract densitometry data. Disclosed invention will not require patient cooperation, locations and size of exit pupils (14, 14a, 14b, 14c) formed by the CGH display (21) will be programmable, it will provide a wide field-of-view and will be much less affected from scattering.

Present invention also addresses the problem of existing simulators not having multifocal IOL assessment capability with patients; since they can only emulate a multifocal IOL design using an artificial eye (30). Disclosed invention employs multiple programmable pinhole generation techniques to simultaneously render content at multiple depths with proper focus cues. Moderate cataracts and refractive lens exchange (RLE) candidates are as such expected to greatly benefit from the disclosed simulator, albeit severe hypermature dense cataracts could have limited success due to serious scattering.

A phantom eye or an artificial eye (30) with an artificial lens such as an artificial cataractous lens, monofocal IOL, or multifocal or trifocal IOL (28), can be used to train the algorithms used in the present invention. In such tests, contrast of the displayed Snellen chart or other visual acuity test patterns may be adjusted. Likewise, halo patterns (32a) and other diffractive visual artefacts (32) similar to those formed by multifocal and trifocal IOLs (28) can be added using algorithms to the displayed patterns, different levels of glare may also be added. Another adjustable feature in the disclosed invention is the distribution of energy along the axial axis. This can display defocus curves matching different multifocal designs as well as create new custom lens designs. Training of the algorithms using the artificial eye (30) improves the post-surgery visual performance predictions of the disclosed invention.

The present invention contains a computation unit (19) where CGH patterns can be selectively adjusted by the ophthalmologists during the test. The visual parameters that are selectively configurable can be selected from a group including the following; visual acuity, side effects such as halo and glare, contrast, defocus curves, or depth perception.

RLE patients do not suffer from cataracts but would like to get rid of eyeglasses. CGH display is the only technology that can present true 3D with all the depth and focus cues. Therefore, disclosed simulator will make full use of holography for the healthy eye and present information that has trifocal, EDOF, or other functions and the associated defocus curves. Disclosed simulator can have a disruptive effect on the RLE surgery market as it will reduce the anxiety of patients who prefer a glasses free lifestyle.

The most prominent reason for patient dissatisfaction after surgery is the side effects of IOLs, for simulating which there is no simulator available in the market. Disclosed simulator will have software modules for contrast sensitivity, halo and glare artefacts that form around the light sources, and dysphotopsia (unwanted images), which are mainly due to diffractive effects and edge effects of the multifocal lens surface profile. The CGH computation algorithms used to simulate the side effects can be trained using machine learning algorithms using the artificial eye (30) models.

Disclosed invention possesses the novel capability of representing a wide array of optical abnormalities/disorders using computer generated holography. CGH modulates the amplitude and phase of incident light using an SLM (39) and all of the visual information and cues required by the eye can be reconstructed. Any lens effect can be realistically visualized by replicating its hologram/interference pattern on the SLM (39). Most of the optical disorders/defects of the eye including refractive, diffractive, and geometric errors can be naturally simulated.

Disclosed invention proposes a vision simulator in the form of a head-worn device (18). The architecture for one eye module of the head-worn device (18) comprises a point light source (38), a phase-only spatial light modulator (SLM) (39), optical components (40), exit pupil plane (15) and pupil tracking cameras (22a, 22b) and a computation unit (19). The simulator in the disclosed invention utilizes a partially coherent point light source that illuminates the SLM (39). Spatially coherent diverging beam that is generated by a point source (38) is collimated with a lens before illuminating the SLM (39). The light gets modulated via SLM (39) and create phase modulated beam (35) and at least one exit pupil beams (16) at the exit pupil plane (15), which is substantially overlapping with the eye pupil (13) of the user. The propagated light rays reach the eye (pupil plane) once they are reflected from the beam splitter (36). This optical architecture provides the correct ray angles from virtual objects that are encoded in the phase only holograms. The modulated waves that are reflected via beam splitter (36) propagate and enter the eye pupil to form the retinal image of the virtual object.

Disclosed invention proposes a more accurate postoperative visual accuracy (VA) prediction. Present invention employs a pupil tracker, which will automatically find the relatively clear areas of the cataractous lens using the cataract densitometry data. Disclosed invention does not require patient cooperation, wherein locations and size of exit pupils (14) formed by the CGH display (21) will be programmable, and provides a wide field-of-view and is much less affected from scattering.

Disclosed invention also allows highly accurate assessment of multifocal IOLs before cataract surgery. Existing solutions in the art do not have multifocal IOL assessment capability with patients; they can only emulate a multifocal IOL design using an artificial eye (30). Disclosed invention employs multiple programmable pinhole generation techniques to simultaneously render content at multiple depths with proper focus cues. Even in hypermature dense cataract cases, coherent beam in the CGH display may have less severe scattering artefacts compared to the incoherent beams in regular displays. The disclosed invention can reduce the impact of scattering using additional phase correction terms in the hologram computation.

Disclosed invention also enables simulation of potential side effects due to cataract surgery. A major reason for perceived unhappiness for patients after cataract surgery is the side effects of intraocular lenses. In the state of the art, there exists no simulator that may accurately account for simulating potential side effects of such lenses. Disclosed simulator comprises software modules for contrast sensitivity, halo and glare around the light sources, and dysphotopsia (unwanted images), which are mainly due to diffractive effects and edge effects of the multifocal lens surface profile.

In a more elaborated manner, disclosed invention takes advantage of eye-box steering. Exit pupil or eye-box steering is a technique that is commonly used by holographic near-eye display architectures to overcome the fundamental trade-off between the field-of-view and eye-box size. Therefore, there are techniques to enlarge the eye box by changing the focus using a dynamic mirror or by creating multiple focal spots using a holographic optical element. Known focus steering methods require either mechanical motion or fixed positions of the multiple focal spots. On the other hand, CGH algorithms allow the disclosed invention to produce the effect of various optical components on the display system by computationally embedding their wave shaping properties in the objective wave calculation. Therefore, CGH remains as the only technology that provides complete computational control over the exit pupil (eye-box) size, location, and shape.

The full complex hologram can be calculated using the Fresnel Space Propagation and a phase-only hologram can be obtained using iterative Fourier transform algorithms. Disclosed invention proposes an optical configuration is illustrated in FIG. 7, where the exit pupil corresponding to the generated hologram can be placed between the 0^{th} and the 1^{st} diffraction orders. The horizontal and vertical distances of the exit pupil in 2D from the 0^{th} order location of each color can be determined by solving the grating equation:

$${d}_{x} = \frac{{m}_{x} λ f}{a} , {d}_{y} = \frac{{m}_{y} λ f}{a}$$

where dₓ=d_{y}=5.25 mm is the pixel pitch, mₓ and m_{y} are the diffraction order numbers in the horizontal and vertical axis, λ is the wavelength, and f is the focal length of the lens. For placing the center of the hologram beam between the 0^{th} and 1^{st} diffraction orders, mₓ and m_{y} can be selected any number between 0 and 1. For the display system of the disclosed invention, f is approximately 50 mm, and the pixel pitch of our phase-only spatial light modulator (SLM) is 4.5 μm. For m=m_{y}=1, the maximum value of dₓ=d_{y}=5.25 mm. While using the Iterative Fourier Transform Algorithm for phase hologram computation, about 5% of the area is allocated to phase noise; therefore, the available region for the eye-box placement is approximately 5 mm-by-5 mm.

In order to steer the eye-box anywhere in the square area between the 0^{th} and 1^{st} diffraction orders in the 1^{st} quadrant, one embodiment of the disclosed invention adds a linear grating phase term in x and y-axis with periods proportional to mₓ and m_{y}. A different value of m has to be calculated for the horizontal and vertical axis and for each color. According to an embodiment, for blue wavelength (473 nm), the exit pupil location corresponds to m=m_{y}=0.5. Said area is divided into 5-by-5 subregions to form 25 different exit pupils where each exit pupil (14) carries exactly the same scene information. Individual eye-boxes are separated with 0.2 mm. While multiple exit pupil (14) locations can be activated simultaneously, only one of the exit pupils are activated at a time and sequentially moved to each of the 25 exit pupil (14) positions during the clinical trials in order to utilize healthy sections of the patient's cataractous pupil.

Note that, when multiple exit pupils (14) are activated simultaneously, one can also embed different holograms containing different parallax of the scene into each exit pupil (14). While such an approach can improve depth perception, it is not trivial as one has to also control the coherent interferences between different exit pupil beams (16) at the retina creating image artefacts.

The prevalence of refractive problems among patients raises difficulties to profile the clear spots on the cataractous lens. It is imperative to eliminate the effect of the first-order aberrations caused by the crystalline lens shape since these problems prevent disclosed invention's instrument from providing a sharp virtual image for the patient. The most common aberrations among refractive errors are hyperopia (farsightedness) and myopia (nearsightedness).

Hyperopia and myopia are conditions that cause an image of an object to become unfocused on the retina. Myopia is a condition in which, opposite of hyperopia, an image of a distant object becomes focused in front of the retina. These refractive errors may be corrected with various prescription glasses or contact lenses specifically designed to counteract their effects. Nearsightedness (myopia) is corrected using a concave lens which is placed in front of a myopic eye, moving the image back to the retina and making it clearer. On the other hand, long-sightedness (hyperopia) is corrected using a convex lens, which is placed in front of a hypermetropic eye, moving the image forward and focusing it correctly on the retina. In other words, the focal planes of the environments are adjusted according to patients' measured diopter values of the refractive problems.

Refractive errors are represented in a specific notation in the eyeglass prescription, e.g., +2.00+1.50×180. The first number represents the spherical correction in diopters. A positive sign in front of the first number indicates farsightedness, whereas the negative sign indicates nearsightedness. The second number represents the cylindrical correction in diopters, which indicates the amount of lens power needed for astigmatism. The last number indicates the orientation of astigmatism. 90 corresponds to the vertical meridian of the eye, whereas 180 corresponds to the horizontal meridian.

The elimination procedure in the disclosed invention for hyperopia and myopia can be described as a graphical replication of the effect of the prescribed eyeglasses. The near point of a human eye, defined to be 25 cm, is the shortest object distance that a healthy eye can accommodate or to image onto the retina. As described in the virtual scene creation, the nearest depth plane is described as 25 cm for an ideal case, whereas the digital version of the Snellen chart is located at the farthest depth plane, which is located at 4 m. Depending on the refractive problem, these virtual depth planes are adjusted during disclosed invention's plane discretization procedure of CGH calculation. Depth values that are retrieved from a rendering software are adjusted to replicate the patient's prescribed eyeglasses effect on these planes.

Disclosed invention takes advantage of Computer Generated Holograms (CGH). CGHs are phase patterns that offer the possibility of creating wave-optical display systems that are under complete computer control. CGH calculation in the disclosed invention involves four major steps: content generation, focal plane discretization, object wave computation, and 3D image reconstruction. The desired virtual content is formed, rendered perspective frames are discretized into multiple focal planes with respect to their depth map values. Once the optical properties of the system are defined, the next step computes the object wave of the scene planes with respect to Fresnel Space Propagation. The complex valued objects wave that are calculated to represent the 3D scene. The CGH system generally uses three methods for encoding: the amplitude holograms where the amplitude of the reference wave is modulated, phase holograms which modulate its phase and complex holograms where both amplitude and phase are modulated. In order to display the computed holograms on the phase-only SLM, complex valued hologram frames are phase mapped in the disclosed invention. As a final step, once the encoded CGH has been acquired to reproduce the 3D image of the scene, it can be displayed on a beam shaping device that is explained hereinafter.

In another embodiment, phase CGH patterns are computed using machie learning algorithms and algorithms that are trained using camera-in-the-loop training algorithms.

A head-worn device (18) as a vision simulator is offered in one embodiment of the disclosed invention. The head-worn device (18), as it shown in FIG. 1, has a compact form due to its simple optical architecture. The architecture for one eye module of the head-worn device (18) consists of a point light source (38), a phase-only SLM (39), optical components (40), exit pupil plane and pupil tracking cameras (22) and a computation unit (42). The simulator utilizes a point light source (38) that illuminates the SLM (39). Spatially coherent diverging beam that is generated by a point source is collimated with a lens before illuminating the SLM (39). The light gets modulated via SLM (39). The propagated light rays reach the eye (pupil plane 15) once they are reflected from the beam splitter (36). This optical architecture provides the correct ray angles from virtual objects that are encoded in the CGH frames. The modulated waves that are reflected via beam splitter propagate and enter the eye pupil to form the retinal image of the virtual object.

The point light source (38) can be red, green, and blue lasers or RGB LEDs. While LEDs have limited spatial coherence, they can still produce depth effect and focus blur effect in the virtual images. For a laser-based device in the disclosed invention, the operating wavelengths of the point light source can be selected as 473 nm for blue, 532 nm for green and 632 nm for red color. The grating equation represented previously is wavelength dependent. For different wavelengths, the required m value for pupil steering differs. One can calibrate the setup with respect to blue wavelength since it is the smallest wavelength in the visible spectrum and defines the maximum steerable area in the exit pupil plane. The desired diffracted order is in between m=0 and m=1 diffraction order for blue wavelength. The corresponding order for green and red are adjusted accordingly, i.e., if desired order is at m=0.5 diffraction order for blue, it is m=˜0.4 for green and m=˜0.3 for red. The grating pattern period is adjusted accordingly for each color hologram to keep the wavelength to grating-period ratio constant.

The pupil tracking cameras are placed in front of the beam splitter to align the patient pupil with the exit pupil (14) of the holographic head-worn device (18). Pupil tracking unit (37) contains two individual camera units: one visible range camera (22a), one infrared (IR) range camera (22b) with IR LEDs (34). IR LEDs (34) that operate at 850 nm illuminate the pupil while the pupil tracking algorithm determines the location and the center of the eye pupil (13) from the frames captured via IR camera (22b). If IR LED and camera are aligned with the optical axis of the eye, a bright pupil reflection will appear on the camera image, if the IR LED is off-axis a dark pupil will appear on the camera image. The visible ranged camera (22a) tracks diffraction orders (17) that are formed on the pupil plane. Exit pupil location is estimated with respect to the 0^{th} and 1^{st} order diffraction order (17) locations.

Disclosed invention takes advantage of three-dimensional scenes generatable by rendering programs. Such a virtual scene is formed by two depth planes which provide the perspective and depth map data as raw rendered frames to the CGH algorithm. To comply with the real eye examination scenario, a graphically constructed Snellen chart is used for far-plane content whereas the near-plane only contains the surrounding box of the Snellen chart. In this way, the patient experiences an illusion of the real eye examination scenario. According to such an embodiment, far plane and near plane are formed at 400 cm and 25 cm respectively.

Disclosed invention proposes a near-eye ophthalmic simulation device suitable for use before cataract and/or refractive lens exchange operations.

According to one aspect of the disclosed invention, said near-eye ophthalmic simulation device comprises a pupil tracking unit (37) to detect the position of an eye pupil (13).

According to one aspect of the disclosed invention, said near-eye ophthalmic simulation device comprises a computer-generated holographic display (21) that can form at least one exit pupil (14) wherein the each of the at least one exit pupils (14) are independently sized and positioned relative to the position of said eye pupil (13) wherein each of the at least one exit pupil (14) is configured to create a projected pattern on the retina (11) of the viewer and carries visual information configured to simulate post-operative vision, wherein at least one visual parameter is selectively configurable within said simulator.

According to one aspect of the disclosed invention, said at least one visual parameter selectively configurable is selected from a group including the following; visual acuity, side effects such as halo (32a) and glare (32b), contrast, defocus curves, or depth perception.

According to one aspect of the disclosed invention, an exit pupil beam (16) through said each exit pupil (14) carries visual information corresponding to at least one perspective hologram (44).

According to one aspect of the disclosed invention, said at least one perspective hologram (44) is configured to be either two-dimensional or three-dimensional.

According to one aspect of the disclosed invention, said beam through said each exit pupil beam (16) is relayed using a different color or wavelength, whereby interference between perspective holograms (44) is avoided.

According to one aspect of the disclosed invention, said device is configured such that different perspective holograms (44) create different images on the retina (11) and appear at a predetermined focus distance.

According to one aspect of the disclosed invention, said device is configured such that holograms with objects at multiple depths can be rendered simultaneously using CGH display (21) and at least two of said at least one exit pupils (14).

According to one aspect of the disclosed invention, said device is configured to correct for refractive errors including astigmatism using CGH algorithms.

According to one aspect of the disclosed invention, said device is configured to correct for corneal aberrations using CGH algorithms.

According to one aspect of the disclosed invention, said device is configured to align the at least one exit pupil with the relatively clear areas of the cataractous lens, which are determined using cataract densitometry or pupil tracking unit (37) or other measurement means.

According to one aspect of the disclosed invention, said device is configured such that exit pupil beams (16) from said at least one exit pupil (14) does not interfere with that of another exit pupil (14), whereby coherent interference is avoided.

According to one aspect of the disclosed invention, said device is configured such that the size of said at least one exit pupil (14) is smaller than 2.0 mm, preferably 1.2 mm.

According to one aspect of the disclosed invention, said device is configured such that said at least one exit pupil (14) is displayed in a time-sequential manner.

According to one aspect of the disclosed invention, said ophthalmic simulation device is a binocular device configured to display holographic stereograms to simulate three-dimensional vision.

What is claimed is:

1. A near-eye ophthalmic simulation device suitable for a use before cataract and/or refractive lens exchange operations, comprising;
a pupil tracking unit to detect a position of an eye pupil,
a computer-generated holographic display to form at least one exit pupil, wherein each of the at least one exit pupils is independently sized and positioned relative to the position of the eye pupil;
wherein each of the at least one exit pupil is configured to create a projected pattern on a retina of a viewer and carries a visual information configured to simulate a post-operative vision, wherein at least one visual parameter is selectively configurable within a simulator, and,
the near-eye ophthalmic simulation device is configurable such that the at least one exit pupil is aligned with relatively clear areas of a cataractous lens, the relatively clear areas being determined using measurement means comprising a cataract densitometry or a pupil tracking unit.

2. The near-eye ophthalmic simulation device according to claim 1, wherein the at least one visual parameter selectively configurable is selected from the group consisting of a visual acuity, side effects such as a halo and a glare, a contrast, defocus curves, and a depth perception.

3. The near-eye ophthalmic simulation device according to claim 1, wherein each of exit pupil beams through the at least one exit pupil carries the visual information corresponding to at least one of different perspective holograms.

4. The near-eye ophthalmic simulation device according to claim 3, wherein the at least one of the different perspective holograms is configured to be either two-dimensional or three-dimensional.

5. The near-eye ophthalmic simulation device according to claim 3, wherein a beam through each of the exit pupil beams is relayed using a different color or wavelength, whereby an interference between the different perspective holograms is avoided.

6. The near-eye ophthalmic simulation device according to claim 1, wherein the near-eye ophthalmic simulation device is configured such that different perspective holograms create different images on the retina and appear at a predetermined focus distance.

7. The near-eye ophthalmic simulation device according to claim 1, wherein the near-eye ophthalmic simulation device is configured such that holograms with objects at plurality of depths are rendered simultaneously using a computer generated holographic (CGH) display and at least two of the at least one exit pupil.

8. The near-eye ophthalmic simulation device according to claim 1, wherein the near-eye ophthalmic simulation device is configured to correct for refractive errors including astigmatism using CGH algorithms.

9. The near-eye ophthalmic simulation device according to claim 1, wherein the near-eye ophthalmic simulation device is configured to correct for corneal aberrations using CGH algorithms.

10. The near-eye ophthalmic simulation device according to claim 1, wherein the near-eye ophthalmic simulation device is configured to align the at least one exit pupil with the relatively clear areas of the cataractous lens by shaping and steering at least one beam of a light or using mechanical adjustments.

11. The near-eye ophthalmic simulation device according to claim 3, wherein the near-eye ophthalmic simulation device is configured such that the exit pupil beams from the at least one exit pupil do not interfere with each other, whereby a coherent interference is avoided.

12. The near-eye ophthalmic simulation device according to claim 1, wherein the near-eye ophthalmic simulation device is configured such that a size of the at least one exit pupil is smaller than 2.0 mm.

13. The near-eye ophthalmic simulation device according to claim 1, wherein the near-eye ophthalmic simulation device is configured such that the at least one exit pupil is displayed in a time-sequential manner.

14. The near-eye ophthalmic simulation device according to claim 1, wherein the near-eye ophthalmic simulation device is a binocular device configured to display holographic stereograms to simulate a three-dimensional vision.

15. A method of computer-generated holography suitable for a use in simulating a post-operative outcome in candidates of cataract and/or refractive lens exchange operations, comprising steps of:
a first multifocal assessment, wherein a person is subjected to first multi-depth, three-dimensional holographic images with first adjustable defocus curves for a first near, intermediate, and far vision,
a visual acuity and retinal assessment, wherein the person is subjected to a visual acuity chart, an astigmatism correction, an aberration correction, and an assessment of a foveal vision,
a contrast sensitivity assessment, wherein the person is subjected to the visual acuity chart and images with adjustable dimming and contrast levels,
a side-effect assessment, wherein the person is subjected to a set of artificially generated images with diffractive and/or multifocal surface artefacts such as a halo and a glare are superposed thereon with varying levels of a severity, and
a second multifocal assessment, whereby the person is subjected to second multi-depth, three-dimensional holographic images with second adjustable defocus curves for a second near, intermediate, and far vision.

16. The near-eye ophthalmic simulation device according to claim 12, wherein the size of the at least one exit pupil is 1.2 mm.

17. The near-eye ophthalmic simulation device according to claim 2, wherein the near-eye ophthalmic simulation device is configured such that different perspective holograms create different images on the retina and appear at a predetermined focus distance.

18. The near-eye ophthalmic simulation device according to claim 3, wherein the near-eye ophthalmic simulation device is configured such that the different perspective holograms create different images on the retina and appear at a predetermined focus distance.

19. The near-eye ophthalmic simulation device according to claim 4, wherein the near-eye ophthalmic simulation device is configured such that the different perspective holograms create different images on the retina and appear at a predetermined focus distance.

20. The near-eye ophthalmic simulation device according to claim 5, wherein the near-eye ophthalmic simulation device is configured such that the different perspective holograms create different images on the retina and appear at a predetermined focus distance.
